(19) United States
(12) Patent Application Publication
(10) Pub. No.: US 2005/0222617 A1
(43) Pub. Date: Oct. 6, 2005

(54) Functionally colored surgical needle

(76) Inventor: Steven J. Ororz JR., Oregon, OH (US)

Correspondence Address: Charles F. Schroeder, 7344 Country Commons Lane, Sylvania, OH, 43560, US

(21) Appl. No.: 10/818,282

(22) Filed: Apr. 5, 2004

(51) Int. Cl. A61B 17/04

(52) U.S. Cl. 606/223

(57) ABSTRACT

A surgical needle which has an environmentally contrasting color along its length to make it more distinctly visible in surgical procedures to facilitate greater accuracy in use of the needle and to impart safety against needle sticks and accidental retention in the body of patients operated on. One, two or more colors can be provided in zones along the needle length to promote greater visibility and performance of specific functions for which the needle is designed.

This invention is a surgical needle provided with color which is contrasting to the color of flesh and blood confronted in surgery procedures.

### BACKGROUND AND BRIEF DESCRIPTION OF THE INVENTION

Surgical needles are usually of metal with a mirror-like finish such as stainless steel which when used under the usually well lit, high intensity light conditions such as in operating rooms are highly reflective of their environment. When inserted into flesh in the vicinity of blood confronted during surgery they reflect the surrounding color of flesh and blood and become less visible or practically invisible in areas where the needle is inserted and directed to a withdrawal point.

By providing the body of the needle with a color which is contrasting to the environment, such as blue, yellow or green, it is clearly more than usually visible, in the environment. A surgeons manipulation or maneuvering of a contrasting colored needle which is clearly more than usually visible can be more precise and can result in greater accuracy in performance of a surgical procedure. For example when a surgical needle used for stitching a wound is provided an environmentally contrasting color, such as green, it can be more clearly seen such as when the tip of the needle is brought to the surface across a wound for withdrawal and reinsertion.

Thus an object of the invention is to make it possible for surgical needles to be used more precisely and accurately during the performance of surgical procedures.

Another object is to make the use of surgical needles more safe in handling outside the wound by making them more visible and thereby lend to reducing the number of accidental sticks.

Still another object of the invention is to make the surgical needles more visible during surgery so as to reduce the possibility of loss within wound and result in becoming a retained item left in a patient unintentionally as has been experienced at times during closing procedures following operations.

Contrasting colors to the red blood environment in which the needles are most frequently used are colors not naturally present in the body or shades such as blue, green and yellow or mixtures thereof as well as black. Since the surgical rooms are usually lit with a fluorescent light, coloring the needle with fluorescent matter would be advantageous in that the color would be more visible in such light phosphorescent matter or iridescent coloring matter can also be used as an advantage to make the needles more visible for surgical procedures.

A surgical needle can also be provided with two different colors along its length such as by providing one color at its tip and another along its body. Thus the tip would be more clearly visible when emitted from the flesh for withdrawal such as in stitching as wounds.

Where the needle has one or two cutting edges the portion which is provided with the cutting edges can be one color with another color at its tip. The colored regions could thus also be used as a measuring means to indicate more clearly where the cutting region is present along the length of the needle.

The body of the needle can also be provided with more than two or more colors along its length to indicate regions to be used for functions to be performed. For example one color can be provided along the length where: one cutting edge is provided and another color where more than one cutting edge is provided and thereby promote proper use of the needle for the functions and in the regions for which it was designed.

Other objects and features which are believed to be characteristic of the invention are set forth with particularity in the appended claims.

### BRIEF DESCRIPTION OF THE DRAWINGS

FIG. 1 is an illustration of a surgical needle of the type which is provided an environmentally contrasting color for the environment in which it is used.

FIG. 2 is representative of the manner of use of the surgical needle of FIG. 1 in stitching a wound.

FIG. 3 illustrates how a needle such as shown in FIG. 1 can be provided two or three different colors along its length.

FIG. 4 illustrates a number of different shaped surgical needles for which the concept of the present invention can be adapted.

### DESCRIPTION OF THE INVENTION IN DETAIL

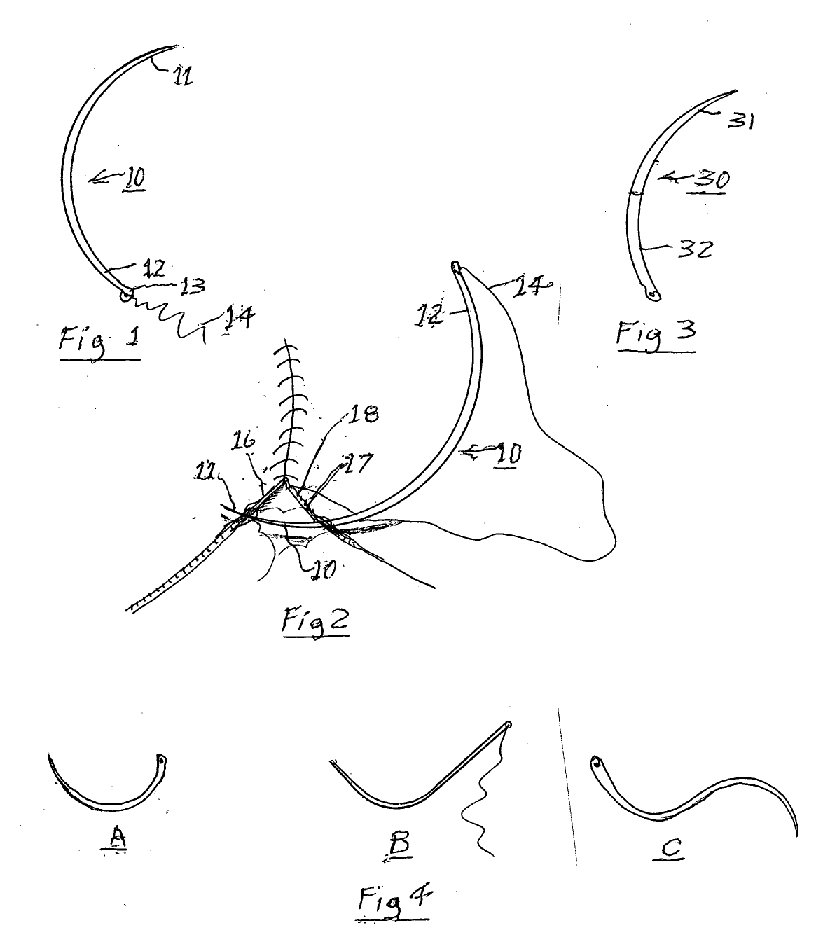

Turning to the drawings in detail, FIG. 1 is representative of a common shape of a surgical needle among a wide spectrum of different shaped surgical needles commercially available. As illustrated the needle in this case representative of an arc shaped needle which is colored according to the present invention. Generally the needle is a thin crescent moon shaped needle or a bow shaped needle such as might be the shape of a bow in a bow and arrow assembly. The needle 10 has a tip 11 and an opposite base end 12 with an eye 13 for fastening of a stitching thread 14 thereto.

FIG. 2 illustrates the needle of FIG. 1 in use in which circumstance needle 10 is inserted on one side 16 of a cut or a wound 18 and extends to the other side 17 of the wound where the tip 4 of the needle emerges. When the tip is sufficiently exposed it can be grasped to pull its body across the wound 18 with the stitching thread pulled thereafter to bridge the wound and effect the drawing of the two sides 16 and 17 together. As can be seen in FIG. 2 when the conventional needle of stainless steel having a reflective surface is positioned as illustrated the needle is in a position to reflect the color of the environment, usually a blood red environment and can become somewhat lost in the background so that it is not clearly visible.

When as proposed the needle is provided with a color such as blue, yellow, green or violet, or the color of fluorescent, phosphoresent or iridescent matter it will be more clearly visible in the environment so that the insertion of the tip from one side 16 to the other side 17 can be more effective. In other words, the emergence of the tip 11 becomes more clearly visible and accuracy of the stitching procedure can be greatly enhanced. Besides greater accuracy or precision in effecting the procedure, safety in the use of the needle is also enhanced since it is less likely to result in a colored needle stick by reason of the visibility of the needle point. As seen in the illustration in FIG. 2 the needle point by reason of having a contrasting color can be seen more readily even with a short emergence from the flesh in which case a grasping tool might be utilized sooner than if the point were not as readily seen in the environment upon the short emergence.

FIG. 3 is another arrangement of the invention in which a needle 30 is provided with two colors along its length. One color being at the tip end 31 and another color at the trailing end 32. Such two colors might be contrasting such as being a yellow or green at the tip end and a darker color such as blue at the trailing end. In this arrangement a black section also might be provided at either end of the needle. Further in this regard three or more colors if predetermined length segments can be provided along the needle length including the possibility of using them for measuring purposes.

FIG. 4 illustrates four different styles of curvalinear shaped needles that are conventionally available from supply houses. Needle A is a half circle shaped needle while needle B has a long section with its tip end being a short bent section. FIG. 4C might be described as a stretched out S-shaped needle. These different shaped needles are illustrated to show the wide range of different shaped needles commercially available in which the invention can be advantageously embodied. Some needles are provided with a sharp cutting edge between the base and the tip which can be provided with one color such as green or yellow while the two ends are provided another color or the ends can be provided with colors different from each other.

As still another arrangement, the length of the different colored zones, such as the tip end and the tail end with a body portion inbetween, can be used functionally to indicate the depth of penetration and or the amount of the tip end emerging from the stitched region for adequate grasping and drawing of the needle from the flesh to effect tightening of a wound.

In view of the foregoing it will be understood that many variations of the arrangement of the invention can be provided within the broad scope of principles embodied therein. Thus, while particular preferred embodiments of the invention have been shown and described it is intended by the appended claims to cover all such modifications which fall within the true spirit and scope of the invention.

1- A surgical needle for use in a surgical environment comprising a needle having a longitudinal curvalinear body extending to a sharp tipped insertion end, said needle having at least a portion of its length of color distinctly and contrastingly visible in the usual blood red environment in which it is used.

2- A surgical needle as set forth in claim 1 in which the full length of the needle is of one color.

3- A surgical needle as set forth in claim 1 in which the opposite ends of the needle are of the same color spaced from each other with a different color on the inbetween body portion.

4- A surgical needle as set forth in claim 1 in which the needle is provided at least two distinctly and contrastingly visible colors, the tip end being of one such color and the opposite end being of another color.

5- A surgical needle as set forth in claim 1 in which each distinctly visible color is selected from a group consisting of blue, green, violet and yellow, mixtures thereof and different shades of each.

6- A surgical needle as set forth in claim 1 in which each distinctly visible color is provided by matter selected from a group consisting of fluorescent, phosphorescent or iridescent matter.

7- A surgical needle as set forth in claim 1 in which the color is black.
